(19) United States
(12) Patent Application Publication
(10) Pub. No.: US 2002/0197640 A1
(43) Pub. Date: Dec. 26, 2002

(54) Diagnostic phenotype assay for engineered cells and tissues

(76) Inventors: Ichiro Nishimura, Los Angeles, CA (US); Keisuke Iida, Los Angeles, CA (US)

Correspondence Address: OPPENHEIMER WOLFF & DONNELLY, 38th Fl., 2029 Century Park East, Los Angeles, CA, 90067, US

(21) Appl. No.: 10/174,658

(22) Filed: Jun. 19, 2002

Related U.S. Application Data
(60) Provisional application No. 60/299,910, filed on Jun. 21, 2001.

(51) Int. Cl. C12Q 1/68; C07H 21/04

(52) U.S. Cl. 435/6; 536/23.5; 536/23.2

(57) ABSTRACT

The present invention provides an improved method for assessing, monitoring and/or determining the phenotype of cells and tissues. One aspect of the present invention is a method of fabricating phenotype specific gene (PSGs) and house keeping gene (HKGs) targets onto a microarray. Another aspect of the present invention provides a composition containing PSGs and HKGs as targets for high throughput assays including microarray analyses. Another aspect of the present invention is accessing, monitoring and/or determining the phenotype of tissue engineered cells derived from stem cells including embryonic stem cells, embryonic germ cells, fetal stem cells and adult stem cells by hybridizing cDNA probes to either PSG or HKG targets. These methods employ at least 25 PSG targets and no greater than 5000 HKG targets.

### CROSS-REFERENCES

This application claims priority from Provisional Application No. 60/229,910 by Ichiro Nishimura and Keisuke lida, filed Jun. 21, 2001, and entitled “Diagnostic phenotype assay for engineered cells and tissues,” the contents of which are hereby incorporated by reference.

### BACKGROUND OF THE INVENTION

1. Field of Invention

This invention relates generally to compositions and methods for assessing, monitoring, and/or determining the relative phenotype of cells and tissues.

More particularly, the invention relates to compositions including, but not limited to, a cDNA microarray comprised of at least 25 phenotype sensitive genes (PSGs) and about 96 but not greater than 5,000 commonly expressed house keeping genes (HKGs).

Also, more particularly, the invention relates to methods for assessing, monitoring, and/or determining the phenotype of transplanted cells and tissues derived from tissue-engineered cells.

Specifically, the present invention provides for compositions comprising of at least 25 PSGs including extracellular matrix (ECM) protein gene targets.

Further, the present invention provides for compositions comprising of about 96 but not greater than 5,000 housekeeping genes.

Still further the present invention provides methods for assessing, monitoring and/or determining the phenotype of various tissue-engineered cells by hybridizing microarray targets to fluorescently labeled cDNA probes from RNA of tissue-engineered cells to form an expression profile of genes transcribed in the tissue-engineered cells.

Still further the present invention provides methods to modulate fabrication of microarrays, cDNA probe preparation, and selection of PSG targets dependent on the tissue type and tissue engineering strategies.

2. Description of the Related Art

Until recently, surgeons grafted implants using one of three procedures: 1) an autograft, whereby a piece of tissue is removed from one area of a patient's body and placed in another location; 2) an allograft, whereby a section of tissue from one human is grafted to another human; and 3) a xenograft, whereby a tissue is harvested from another animal species. All three procedures are problematic due to the availability of suitable tissue, immunological reactivity (for allografts and xenografts), and increased stress and high cost to patients.

Alternatively, tissue engineering involves the use of living cells and extracellular components, either natural or synthetic, to develop implantable parts for the restoration, maintenance, or replacement of function. For example, artificial organs and/or tissues grown outside of the body, in the laboratory, are transplanted to the patient suffering from the diseased or defective organ or tissue. These replacement parts typically consist of both cellular tissues and artificial or non-artificial matrices grown ex vivo.

Tissue engineering also requires the isolation and propagation of undifferentiated cells because fully differentiated cells are difficult to proliferate. Research in human developmental biology has led to the discovery of human stem cells (precursor cells that can give rise to multiple tissue types), including embryonic stem cells, embryonic germ cells, fetal stem cells and adult stem cells. Experiments aimed at determining the mechanisms underlying the conversion of stem cells into differentiated cells comprising various organs and tissues of the human body has great promise.

It is postulated that a “master gene” is responsible for determining the fate of the engineered cells and tissues. One study has shown that injection of a “master gene” RNA into embryonic zebrafish cells induced development of those cells to become differentiated heart cells (Reiter J F et al., 1999, Genes and Development, 13(22):2983-95). These types of studies would support that monitoring a “master gene” is a good enough assessment of phenotypic development. However, it has been shown that an assay monitoring expression of the “master gene” alone does not conclusively diagnose the fate and development of engineered cells (Prince et al., 2001, J Cell Biochem 80:424-440). The interaction between cells and the extracellular matrix (ECM) shows that the ECM is no longer dismissed as an inert scaffold, rather ECM is a vital part of cell-cell interactions and tissue maintenance.

ECM is essentially any material produced by cells and secreted into the surrounding medium. Typically ECM is applied to the noncellular portion of animal tissues. In broad terms there are three major components of all ECM: 1) fibrous elements including collagen, elastin or reticulin; 2) link proteins including fibronectin and laminin; and 3) space filling molecules including glycosaminoglycans. The ECM may be mineralized such as in bone, or dominated by tension resisting fibers such as in tendon. Another example of ECM is the basal lamina of epithelial cells.

Again in broad terms, ECM proteins function to modulate cell attachment, growth, migration and spreading. Interactions between cells and the ECM, therefore, play a crucial role in development, differentiation, growth, remodeling, and wound healing. Studies have even shown that cell-ECM interactions regulate gene expression at the transcriptional level during physiological and pathophysiological events (Ashkenas et al., 1996, Dev Biol, 180:443444). Hence, the intracellular interactions between the ECM and that of the cell are essential to understanding transplanted tissue-engineered cells in the patient.

Thus, the ultimate goal of tissue engineering is to understand the critical events underlying growth, development, homeostasis and behavior of cells and tissues at the genomic level during transplantation of stem cells into the patient. In the areas of dental, oral and maxillofacial research, elucidating the molecular and genetic bases of normal and abnormal conditions is a major goal. However, the complex mechanisms of the genetic pathways specific to oral and maxillofacial tissues in both physiological and pathophysiological events are not yet fully understood.

In particular, to understand specific biological pathways associated with the molecular and genetic bases of normal and abnormal dental, oral and maxillofacial cells and tissues, an improved method to assess, monitor and/or determine cell phenotype is a primary goal. Traditional molecular biology studies use one gene/one experiment models. Yet, during cellular differentiation there is the expression of multiple genes at any one time, making standard molecular biology methodologies tedious, laborious and inefficient. Hence, a high throughput assay to assess, monitor and/or determine the levels of expression of many genes is necessary.

### INVENTION SUMMARY

A general object of the present invention is to provide a method to monitor levels of gene expression in cells and tissues.

In accordance with one aspect of the present invention, these and other objectives are accomplished by providing a microarray technology and/or DNA chip technology, which is capable of hybridizing a plurality of nucleic acid targets to a plurality of nucleic acid probes.

In accordance with one aspect of the present invention, these and other objectives are accomplished by providing a cDNA microarray with a plurality of phenotype specific gene (PSG) targets, more particularly, extracellular matrix (ECM) targets.

In accordance with another aspect of the present invention, these objectives are accomplished by providing a focused microarray containing a plurality of phenotype specific gene (PSG) targets.

In accordance with another aspect of the present invention, these objectives are accomplished by providing a composition containing at least 36 PSGs, more particularly, approximately 50 ECM genes including but not limited to human Osteocalcin, rat Osteocalcin, human Osteopontin, rat Osteopontin, human Osteonectin, rat Osteonectin, human Alkaline phosphatase, human Bone morphogenetic protein 7, human Estrogen receptor, human Vitamin D receptor, human Bone morphogenetic protein 2, mouse Core binding factor A1, human Integrin alpha2, rat Integrin beta1, rat Integrin beta3, rat Parathyroid hormone receptor, rat Bone sialoprotein II, human Matrix metalloproteinase 1, human Matrix metalloproteinase 2, human Laminin B1, human Syndecan2, human Chondroitin sulfate proteoglycan 1, human Decorin, human Fibronectin, human Tenascin X, human Collagen type1 alpha1, rat Collagen type1 alpha1, rat Collagen type2 alpha2, rat Collagen type2 alpha1, human Collagen type3 alpha1, human Collagen type4 alpha1, Collagen type4 alpha2, human Collagen type5 alpha1, human Collagen type5 alpha2, human Collagen type6 alpha1, human Collagen type6 alpha3, human Collagen type7 alpha1, rat Collagen type9 alpha1, human Collagen type9 alpha2, human Collagen type9 alpha3, rat Collagen type10 alpha1, human Collagen type11 alpha, mouse Collagen type11 alpha1, mouse Collagen type11 alpha2, human Collagen type12 alpha1, human Collagen type14 alpha1, human Collagen type15 alpha1, human Collagen type16 alpha1, human Collagen type19 alpha1and Apolipoprotein E2.

In accordance with another aspect of the present invention, these objectives are accomplished by providing a composition containing substantially 96 house keeping genes (HKGs) including but not limited to Human alpha-catenin, Human EST2, Human cytochrome c-1, Human uroporphyrinogen III synthase, HPV16 E1 binding protein, Human guanine nucleotide-binding (alpha subunit mRNA), Homo sapiens splicing factor SF3a120, Homo sapiens adenylyl cyclase-associated protein (CAP), Human cytochrome bc-1 complex core protein II, Human platelet-type phosphofructokinase, Homo sapiens deoxyhypusine synthase, Human hnRNP core protein A1, Human coatomer protein (HEPCOP), Homo sapiens phosphatidylinositol 4-kinase mRNA, Human AMP deaminase (AMPD2), Protein Translation Factor SUI1, Homo sapiens alpha centractin, Human Na/H antiporter (APNH1), Human lysosomal glycosylasparaginase (AGA), Human acidic ribosomal glycosylaparaginase (AGA), Human acidic ribosomal phosphoprotein P0, Human capping protein alpha, Human mercurial-insensitive water channel, Human ionizing radiation resistance conferring protein A, Human mitochondrial short-chain enoyl-CoA hydrase, Homo sapiens elongation factor-1-gamma, Homo sapiens catechol-O-methyltransferase (COMT), Human cytoplasmic beta-actin, Human calmodulin, Human 90 kDa heat shock protein, Human elongation factor Tu-mitochondrial, Human U1 snRNP-specific protein A, glycogenin-2 delta, Human Xq28, creatine transporter (SLC6A8), Human beta-tubulin, pulmonary surfactant protein (SP5), Human protein kinase, Homo sapiens cyclin H assembly factor, Human eIF-2-associated p67, Human liver glutamate dehydrogenase, Human superoxide dismutase (SOD-1), Human mitochondrial ADP/ADT translocator, Human eukaryotic initiation factor 2B-epsilon, Human chromatin assembly factor-I p60, Spermidine/spermine N1-acetyltransferase mRNA, Human translational initiation factor 2 beta subunit (eIF-2-beta), Human dihydrolipoamide dehydrogenase, Human cytoplasmic chaperonin hTRiC5, Homo sapiens protein tyrosine kinase (Syk), Human ADP/ATP translocase T, Human ubiquitin-activating enzyme E1 (UBE1), Human cytochrome c oxidase subunit, Human histidyl-tRNA synthetase (HRS), Human topoisomerase I, Human 26S proteasome subunit p97, Human nuclear ribonucleoprotein particle (hnRNP) C protein, Human eukaryotic initiation factor 4Aii, Human lactate dehydrogenase-A (LDH-A, EC 1.1.1.27), Human sterol 27-hydroxylase (CYP27) mRNA, Human glutamate receptor 2 (HBGR2), Human alpha-2-macroglobulin mRNA, Human MRL3 ribosomal protein L3, Human histone H2B.1, Human chaperonin protein (Tcp20), Homo sapiens DNA (cytosin-5)-methyltransferase, Human eukaryotic initiation factor 4A1, Human heterogenous nuclear ribonucleoprotein D (hnRNP D), Human ADP-ribosylation factor 1 (ARF1), Homo sapiens endothelin-1 (EDN1), Human ADP-ribosylation factor, Human glyceraldehyde 3-phosphate dehydrogenase, Human mRNA (HA0643) for ORF, Homo sapiens cadherin-13, Human aminoacylase-1 (ACY1), Human DNA repair helicase (ERCC3), Human mitochondrial 3-ketoacyl-CoA thiolase beta-subunit of trifunctional and Human malate dehydrogenase (MDHA).

In accordance with another aspect of the present invention, these objectives are accomplished by providing a method to monitor and determine the expression of a plurality of genes in transplanted tissue engineered cells and tissues using microarray technologies and/or DNA chip technologies.

In accordance with another aspect of the present invention, these objectives are accomplished by providing a method to monitor and determine the expression of a plurality of genes in transplanted tissue engineered cells and tissues using either a cDNA microarray, a focused microarray, or a tissue-specific microarray containing a plurality of ECM targets.

In accordance with another aspect of the present invention, these objectives are accomplished by providing a method to monitor and determine the expression of a plurality of genes in transplanted tissue engineered cells and tissues using either a cDNA microarray, or a focused microarray, or a tissue-specific microarray containing a plurality of ECM targets and hybridizing to a plurality of probes.

In accordance with another aspect of the present invention, these objectives are accomplished by providing a method to monitor and determine the expression of a plurality of genes in transplanted tissue engineered cells, using either a cDNA microarray, or a focused microarray, or a tissue-specific microarray, containing a plurality of ECM targets and hybridizing to a plurality of probes.

The above described and many other features and attendant advantages of the present invention will become apparent from a consideration of the following detailed description when considered in conjunction with the accompanying drawings.

### BRIEF DESCRIPTION OF THE DRAWINGS

Detailed description of the preferred embodiment of the invention will be made with reference to the accompanying drawings.

FIG. 1 is a flowchart of a cDNA microarray analysis system;

FIGS. 2A-E are photographs of: (A) a computer-controlled, custom-built; (B)commercially available robotic microarrayers; (C) magnified view of the printing tip; (D) a plurality of printing tips; and (E) a cDNA microarray;

FIGS. 3A & B are two cDNA microarrays following hybridization; (A) is a typical microarray; and (B) is a microarray with “comet tails”;

FIG. 4 is a graph showing hybridized fluorescent signal intensities versus time;

FIGS. 5A & B are histograms representing the distribution of Cy3/Cy5 ratios; (A) is a distribution of bone tissue from ovarectomized (OVX) rats as compared to control rats harvested 2 weeks post-surgery; and (B) is a distribution of bone tissue from OVX rats as compared to control rats harvested 4 weeks post-surgery;

FIGS. 6A & B are scatter plots of levels of expression ratios of house keeping genes (HKGs); (A) comparison of RNA samples (Sample 1 and Sample 2) derived from the same origin; and (B) comparison of RNA samples from dissimilar samples (Sample 2 and Sample 3);

FIG. 7 is a scatter plot of levels of expression ratios between mouse humerus and calvaria bones using a microarray containing 36 ECM targets; the inset photo is the respective cDNA microarray.

FIGS. 8A-H are scatter plots of levels of expression ratios of house keeping genes (HKGs) and phenotype specific genes (PSGs) in a comparative experiment between mouse calvaria and mouse (A) calvaria, (B) femur, (C) sternum, (D) bladder, (E) Skin, (F) heart, (G) intestine, and (H) brain tissue.

FIG. 9 are scatter plots of levels of expression ratios comparing adipose derived stem cells from different samples of stromal vascular fractions (SVF) that were cultured in standard medium (CM D12) or osteoblast differentiation medium (OS D12); (A) sample number 67; (B) sample number 69.

### DETAILED DESCRIPTION OF THE PREFERRED EMBODIMENTS

This description is not to be taken in a limiting sense, but is made merely for the purpose of illustrating the general principles of the invention. The section titles and overall organization of the present detailed description are for the purpose of convenience only and are not intended to limit the present invention.

Gene expression analysis has been used for the phenotypic assessment of various tissues, as well as engineered tissue. However, classical gene expression techniques, such as Northern or Southern blot analysis, are limited because they cannot evaluate the expression patterns of multiple genes.

It has been shown that comprehensive profiling of gene expression using microarray technologies provides the ability to measure the expression profile of thousands of genes in a single experiment (Schena et al., 1999, DNA Microarrays: A Practical Approach, Oxford, England: Oxford University Press). Hence, microarray technology is ideal for identifying a cell or tissue phenotype.

In brief, microarrays are orderly arrangements of samples providing a medium for matching known and unknown samples, for example, DNA and/or RNA, based on base-pairing rules and automating the process of identifying the unknowns. The basic principle is that single-stranded complementary nucleic acids in both the known and unknown samples will hybridize to each other to form a duplex. Hybridization of nucleic acids is very selective, sensitive and specific.

In terms of the property of arrayed DNA targets with known identity, there are two variants of DNA microarray technology: 1) the oligonucleotide microarray; and 2) the cDNA microarray.

Oligonucleotide microarrays contain arrays of oligonucleotides generally less than 25-mer in length but can be up to 80-mer in length, and are synthesized either in situ (on-chip) or by conventional synthesis followed by on-chip immobilization. The array is then exposed to labeled DNA probes, hybridized, and the identity/abundance of complementary sequences are determined.

The cDNA microarray is fabricated by printing cloned and amplified cDNAs onto a solid surface. For example, a target cDNA (200 to ˜2,500 bases long) is immobilized to a solid surface such as a glass surface and exposed to a set of probes either separately or in a mixture. Similar to oligonucleotide microarrays, cDNA microarrays are subsequently hybridized with labeled probes, and levels of complementation identified.

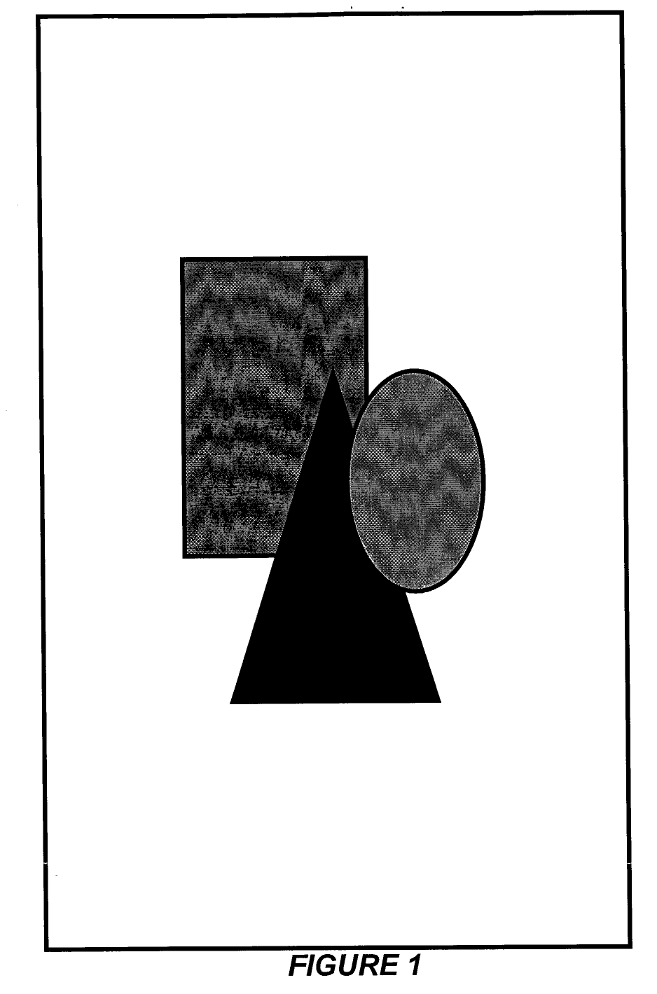

A typical example of a cDNA microarray set-up is shown in FIG. 1. First, the microarray is fabricated onto a solid surface (i.e. glass slide). Then hundreds to thousands of immobilized DNA spots (or “targets”) are directly printed or immobilized onto the microarray or glass surface. Secondly, the targets are simultaneously hybridized with one or more samples (or “probes”) labeled with different fluorescent dyes. After hybridization, the fluorescent signals of each probe bound to individual DNA spots are detected with a confocal laser scanner. Each fluorescent probe is scanned separately and their expression ratios/levels calculated separately. Lastly, the separate images are then combined and pseudocolored using computer software. In short, microarray systems make parallel large-scale gene expression monitoring possible.

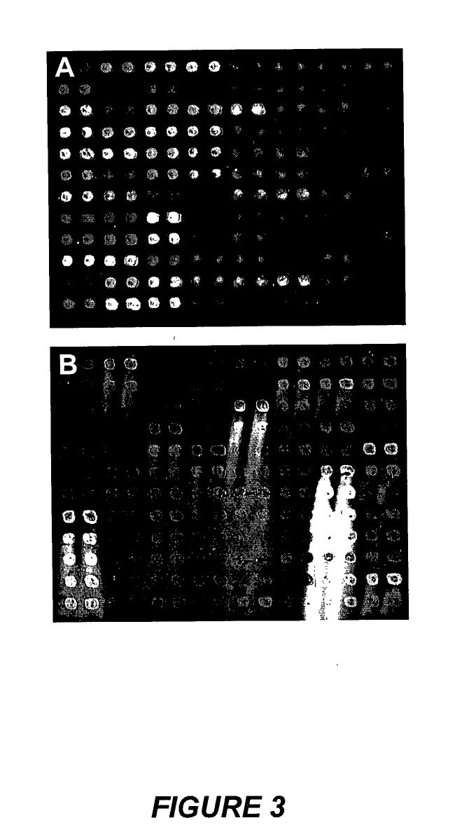

Fabrication of a cDNA microarray (FIG. 1). The first step in cDNA microarray construction is the preparation of cDNA as arrayed targets. An enlarged view of a cDNA microarray can be seen in FIG. 3E. Any double-stranded cDNA, or single-stranded cDNA, can be used for the fabrication of a microarray. In general, cDNAs ranging in length from 0.2 to 2.5 kb are used. To obtain adequate hybridization with a probe cDNA, the concentration of the target cDNA should be as high as 500 ng/μL. Conventional cloning techniques and PCR amplification are usually required for target cDNA preparation. After ethanol precipitation, PCR products are suspended in 3× SSC (20× SSC =17.5% sodium chloride and 8.82% sodium citrate, pH 7.0) and are placed in 96- or 384-well microtiter plates. Target cDNAs are printed onto a poly-L-lysine coated microscope glass slide using a robotic arrayer, as shown in FIGS. 3A & B. The coated surface provides attachment sites for the target cDNA so that it remains bound to the glass surface during hybridization and washing.

Two types of arraying techniques are used for applications of target cDNA to the glass surface: 1) Passive dispensing (FIGS. 3C and D); and 2) Drop on Demand. In passive dispensing(FIGS. 3C and D), the target is loaded into a spotting pin by capillary action and a small volume of the target is transferred to the glass surface by physical contact between the pin and the solid surface. By using a robotic arrayer can print approximately 75,000 genes on a standard 1×3-inch microscope glass slide. The drop-on-demand delivery method is achieved by the adaptation of ink- or bubble-jet technology (Schena et al.,1998, Trends Biotechnol, 16:301-306; Okamoto et al., 2000, Nat Biotechnol, 18:438-441).

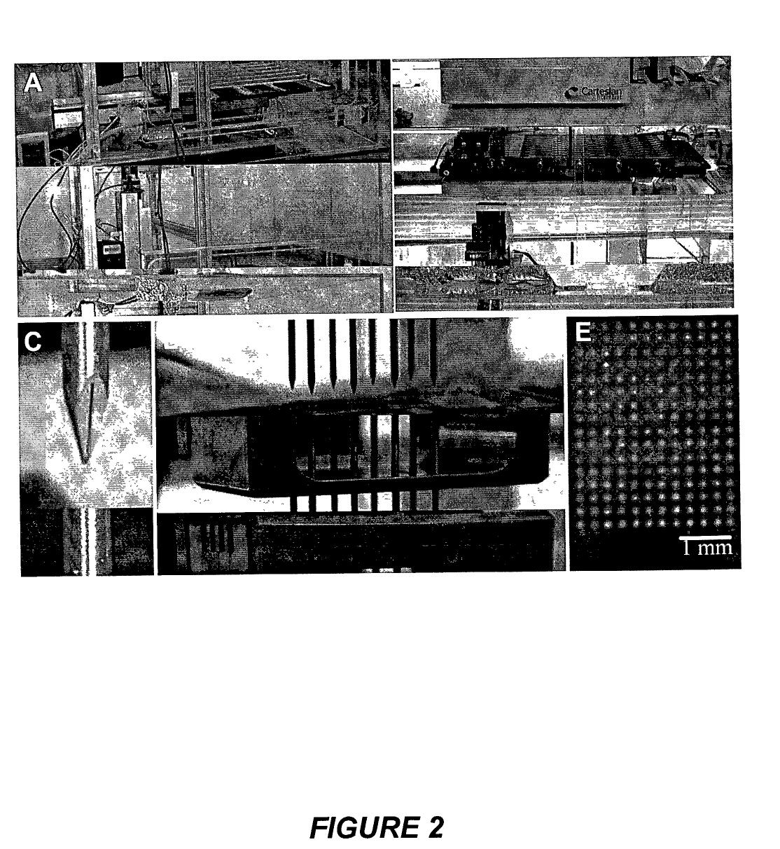

Before hybridization, the microarray requires four steps of post-processing (not shown in FIG. 2): 1) Rehydration and snap drying of the microarray to provide an even distribution of the target cDNA throughout the spot; 2) Ultraviolet cross-linking with 65 mJ of energy to improve the stability of the spotted cDNA; 3) Blocking of the coated glass surface to reduce the non-specific binding of the labeled probe to any remaining free poly-L-lysine; and 4) Denaturation of the printed target cDNA by heating the array with boiling water for 2-3 min.

Step three is essential to minimize the hybridization background noise, since an unnecessary longer period of blocking can cause washing-off of the spotted DNA (or targets). Washing-off of DNA targets creates a phenomenon called “comet tails”. For example, FIG. 3A is a photomicrograph of a typical cDNA microarray with a good blocking protocol and no comet tails. Whereas, FIG. 3B is a similar photomicrograph, except with improper blocking resulting in comet tails. Thus, particular attention must be paid to the blocking process. After post-processing, individual spots on the microarray are usually invisible. Post-processed microarrays are very stable and can also be stored at room temperature for at least six months to probably several years.

Probe preparation and microarray hybridization. Total RNA or mRNA is isolated from tissues or cells to make probes for hybridization. To obtain an adequate fluorescent signal, 15-200 μg of total RNA, depending on the size of the microarray and type of sample, is required for cDNA synthesis and labeling. RNA from test and reference samples is made into cDNA by standard molecular biology protocols well known in the art. The cDNAs are then labeled with two or more different fluorescent dyes. Various types of fluorescent labeling materials are commercially available for use in labeling of probes including Cy3-and Cy5-dUTP or dCTP (Amersham Pharmacia Biotech, Inc., Picataway, N.J.). Cy3-and Cy5-dUTP or dCTP are excellent fluorescent dyes because they are well separated using ultraviolet light emission. Thus, individual dyes can be identified separately. Moreover, Cy3-and Cy5-dUTP or dCTP fluorescent dyes can be directly incorporated into newly synthesizing cDNA during the process of reverse transcription from RNA. Still another advantage of using Cy3and Cy5-dUTP or dCTP fluorescent dyes is they have sufficient brightness for image acquisition processing.

The separately labeled probes are pooled and concentrated. After concentration, successfully labeled probes can be identified by their color. For example, probes labeled with Cy3 and Cy5 typically show light pink and light blue, respectively. Pooled probes of Cy3 and Cy5 are light purple. Probes are then suspended into the hybridization solution containing 3× SSC and 0.5% sodium dodecyl sulfate (SDS) and hybridized to the microarray under a cover slip in a specially designed hybridization chamber. The hybridization chamber is submerged in a 65° C. water bath for 14-20 hours. Alternatively, a mixture of 50% formamide, 3× SSC, 0.5% SDS and 5× Denhardt's solution can be utilized as a hybridization solution. In this case, hybridization should be carried out at 42° C. In addition, supplementation of oligo(dA), Cot-1 DNA, and/or salmon sperm DNA into the hybridization solution is effective for minimizing non-specific hybridization.

Technical Challenges and Alternative Methods.

Alternative techniques for microarray fabrication. In addition to the typical method described above and in FIG. 1, to improve the effectiveness of microarray technology certain features of the technology can be modified and still within the broader scope of the present invention.

For example, Rogers et al. showed that disulfide coupling can be used in the ligation of DNA to increase the stability of the target DNA on the solid support (Rogers et al., 1999, Anal Biochem, 266:23-30). Also, the preparation of target cDNA using the PCR/ligase detection reaction has been shown to increase hybridization sensitivity such that a point mutation is detectable (Gerry et al.,1999, J Mol Biol, 292:251-262; Favis et al., 2000, Nat Biotechnol, 18:561-564).

Modified methods for probe preparation from a small amount of sample. One of the technical challenges of microarray technologies is obtaining distinctive hybridization signals. As described above, DNA microarray hybridization requires relatively large amounts of RNA for cDNA probe synthesis and labeling. However, when provided with only a small amount of sample tissue, or a limited number of available cells, it becomes difficult to prepare enough RNA. If there is insufficient RNA to make cDNA probes, inadequate or undetectable signal intensities of probe and target hybridization results. Therefore, to maintain or improve the fluorescent signal with a concomitant reduction of starting RNA, modified methods for probe preparation and labeling have been applied for microarray technologies.

For example, in vitro transcription (IVT), or antisense RNA (aRNA), or complementary RNA (cRNA) amplification techniques originally developed for gene expression analysis of single cells and extremely small amounts of tissue sample, can be utilized (Kacharmina et al., 1999, Methods Enzymol, 303:3-18). In this method, cDNA synthesis from mRNA is carried out with a specially designed oligo(dT) primer. The oligo(dT) primer contains the bacteriophage T7 RNA promoter sequence [oligo(dT)24-T7]. The cDNA is made double-stranded using conventional techniques well known in the art of molecular biology. Synthesized double-stranded cDNA containing the T7 RNA promoter can then be utilized as a template for aRNA synthesis by the T7 RNA polymerase. The original protocol recommends repeating the amplification procedure to produce a greater concentration of aRNA. By repeating the procedure for two rounds, aRNA is amplified 106-fold greater than the starting material (Eberwine et al.,1992, Proc. Natl. Aca. Sci., 89:3010-3014). This amplified aRNA can be used in microarray assessment, as well as in other methods for gene expression analysis including reverse transcriptase (RT)-PCR.

For microarray hybridization, aRNA amplification is carried out in the presence of biotinylated UTP or CTP. The biotinylated aRNA probe can be hybridized to the microarray and stained with streptavidin-phycoerythrein before or after hybridization to the microarray (Coller et al., 2000, Proc. Natl. Aca. Sci., 97:3260-3265). The detailed protocol for this method is available on the Whitehead/MIT Genome Center's Molecular Pattern Recognition website (http://waldo.wi.mit.edu/MPR/index.html). Alternatively, conventional cDNA synthesis and labeling can also be applied to the amplified aRNA (http://cmgm.stanford.edu/pbrown/).

Mahadevappa and Warrington have demonstrated the effectiveness of the aRNA amplification technique for microarray probe preparation. The investigators improved signal intensities by aRNA amplification with 2.5 μg of starting total RNA (Mahadevappa and Warrington, 1999, Nat Biotechnol, 17:1134-1136). It has also been demonstrated that two rounds of aRNA amplification from 0.01 μg of starting total RNA and Cy dye labeling can produce enough signal for microarray analysis (Wang et al., 2000, Nat Biotechnol, 18:457-459).

Also, since it is possible to analyze the gene expression in a single cell, another approach that can be applied to the microarray technology is integrating aRNA amplification with the whole-cell patch electrode technique (VanGelder et al., 1990, Proc Natl Acad Sci, 87:1663-1667; Eberwine et al., 1992, Proc Natl Acad Sci, 89:3010-3014).

In addition, when aRNA amplification is performed in situ (in situ transcription; IST) on a fixed tissue section or microdissected tissue, the aRNA can be separately amplified in histologically normal and abnormal areas (Tecott et al., 1988, Science, 240:1661-1664; Zangger et al., 1989, Technique, 1:108-117). Therefore, a more accurate comparison of gene expression in histologically different areas within the same tissue section is possible. In fact, the RNA expression patterns in large-and small-sized neurons harvested independently from fixed tissue section by laser capture microdissection can be analyzed by aRNA amplification and DNA microarray as well (Luo et al., 1999, Nat Med, 5:117-122). The results demonstrate the usefulness of the integration of aRNA amplification with the microarray system. Moreover, a combination of aRNA amplification and immunohistochemical staining to compare gene expression profiles between immunologically positive and negative areas in the same tissue is possible.

As another method for increasing the fluorescent signal intensities, amino-allyl reverse transcription (AA-RT) can be used for probe preparation (http://cmgm.stanford.edu/pbrown/). Briefly, cDNA is synthesized from total RNA or mRNA in the presence of amino-allyl dUTP (aa-dUTP, Sigma, St. Louis, Mo.) instead of Cy3- or Cy5-dUTP. The aa-dUTPs incorporated into the synthesized cDNA are coupled with Cy3 or Cy5 monofunctional dye (Amersham Pharmacia biotech, Inc.). Before pooling the two-labeled samples, aa-dUTP is quenched by the addition of hydroxylamine. In the present invention, AA-RT technique results in an increase in fluorescent signal intensities compared with the direct fluorescent dye incorporation method. This technique also has the advantage of reducing the required starting total RNA concentration to less than 10 μg.

When comparing the two different modifications (aRNA versus AA-RT) for increasing cDNA probe starting material, AA-RT method is less effective, but much simpler and easier than aRNA amplification and is the preferred protocol used in the present invention. In contrast, aRNA amplification methods are more effective because greater amounts of cDNA probe can be accomplished with very small quantities of starting RNA. However, aRNA methods involve a long, complex protocol and the use of additional materials, including oligo(dT)24-T7 primer and the IVT kit (Ambion, Austin Tex.).

Data Interpretation and Validation.

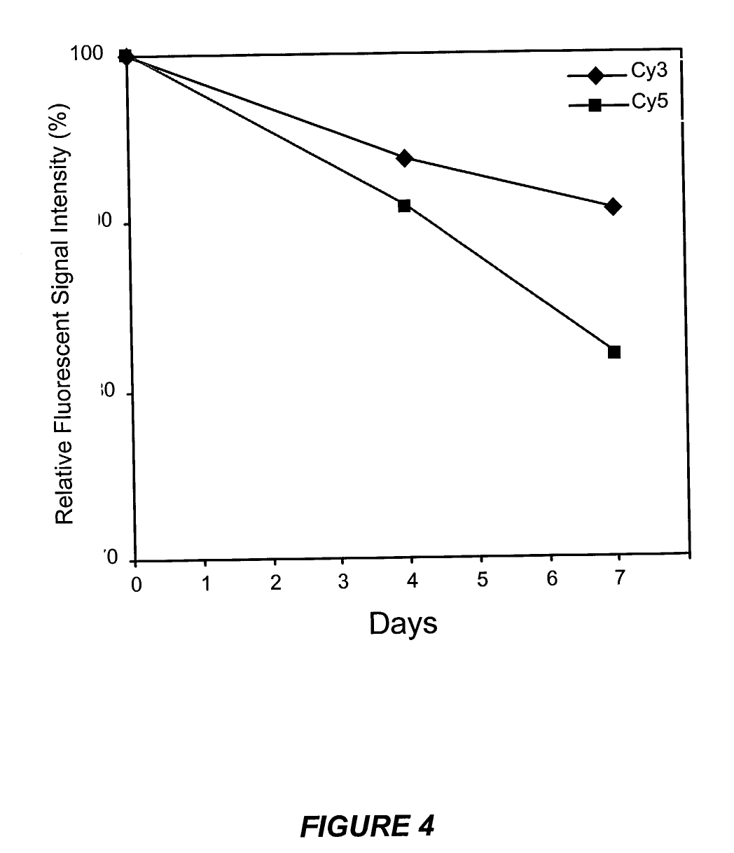

Image scanning. After hybridization and washing, the microarray is scanned using a dual-wavelength confocal laser scanner. To detect Cy3 and Cy5 fluorescent signals, wavelengths of 532 nm and 635 nm are required, respectively. Scanning of the hybridized microarray should be carried out immediately after the washing because the fluorescent dyes lose signal intensity over time. For example, the graph in FIG. 4 shows that although the intensities of the Cy3 and Cy5 dyes immediately after hybridization and washing are the same levels, after seven days they show different fluorescent signal intensities. In particular, the signal intensity of Cy5 is much reduced when compared to the signal intensity of Cy3. Repeated scanning of the microarray also causes a decrease in fluorescent signal intensity, particularly for Cy5 (van Hal et al., 2000, J Biotechnol, 78: 271-280).

The scanned signal intensities of Cy3 and Cy5 should be at the same level for an accurate comparison of two samples. Also, the signal intensities of Cy3 and Cy5 must be adjusted to be as close as possible; because in most cases the starting RNA volumes of the two samples may not be exactly the same. This adjustment can be done using sets of positive control genes including house keeping genes, which are expressed in all cells and code for molecules that are necessary for basic maintenance and essential cellular functions. Although normalization of signal intensities between two samples is usually performed after scanning, adjustment of scanning level makes the normalization process easier.

Measurement and normalization of signal intensities. Separately scanned images of Cy3 and Cy5 signals are transferred into programmed software. Each image of Cy3 and Cy5 is gridded manually, or automatically, to define the areas of the individual spots. Averages and standard deviations of both signal intensities and background noise in individually defined areas are calculated. The difficulty of accurately controlling the starting RNA sample volumes to the same level and the use of two different fluorescent dyes, result in the discrepancy of raw fluorescent signal intensities between two probes. Therefore, a subsequent and very important step is the normalization of the fluorescent signals between two samples.

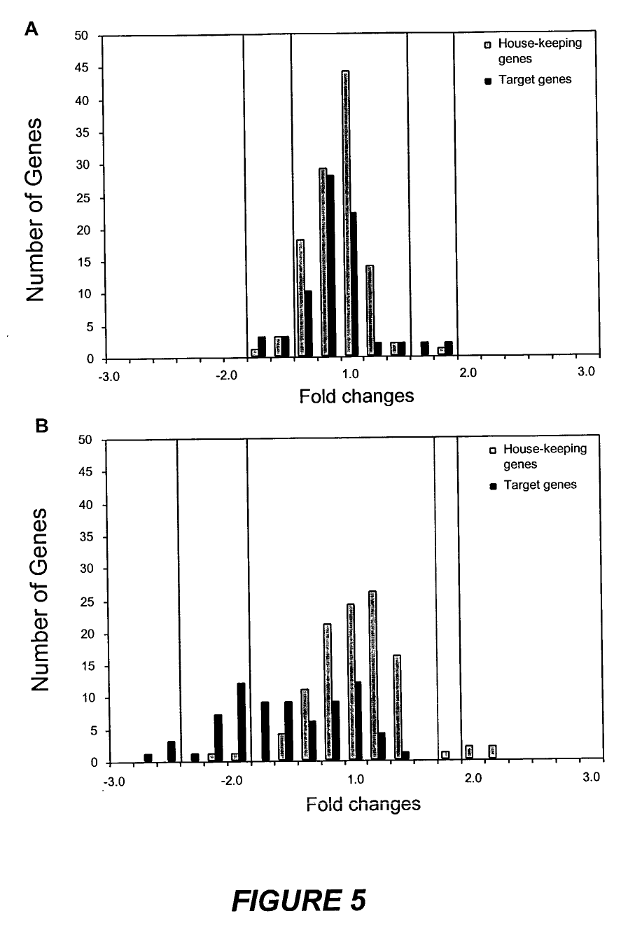

Two different normalization strategies have been used (Duggan et al., 1999, Nat Genet, 21(1 Suppl):10-14): 1) General normalization; and 2) Selected normalization. The general normalization method considers all of the target genes for normalization (Hardwick et al., 1999, Proc Nat Acad Sci, 96:14866-14870; Ross et al., 2000, Nat Genet, 24:227-235). When two probes are derived from closely related samples, the transcriptional levels of many genes are expected to be unchanged. In other words, the Cy3/Cy5 ratios in this situation generally show a “bell-shaped curve” distribution, such as the distribution shown in FIG. 5A. In FIG. 5, continuous lines indicate +/− two-fold changes and interrupted lines indicate 99% Confidence Intervals (CIs). In FIG. 5A, the 99% CIs are included in the range of the +/− two-fold changes. Also, when a large-scale microarray including thousands of genes is used, the distribution of the Cy3/Cy5 ratios also shows a “bell-shaped curve”, similar to that seen in FIG. 5A. Thus, in these instances, the general normalization method is a useful tool.

The selected normalization approach is performed based on the sets of control spots, such as house-keeping genes, which are expressed consistently under most circumstances (Lashkari et al., 1997, Proc Natl Acad Sci, 94:13057-13062; Loftus et al., 1999, Proc Natl Acad Sci, 96:9277-9280; Stephan et al., 2000, Mol Genet Metabol, 70:1018). For example, when divergent samples are compared or a small-scale microarray with hundreds or fewer genes, the transcriptional level may become more varied, resulting in a deviated distribution of the Cy3/Cy5 ratios toward one sample, such as that shown in FIG. 5B. In FIG. 5B, the inconsistency is found between the +/− two-fold differences and the 99% CIs. In these cases of inconsistency, discrepancies between two samples are normalized by using sets of control genes, more particularly, housekeeping genes (HKGs). Table II lists 96 HKGs used in the present invention, however, many other HKGs can be utilized and still keep within the broader scope of the present invention.

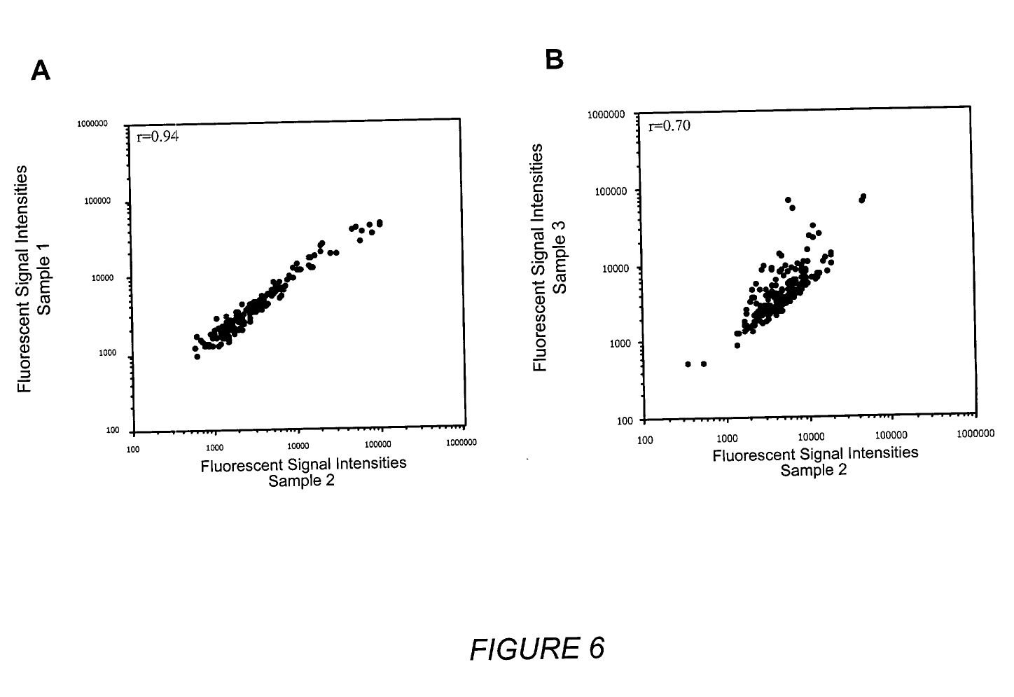

The distribution of the Cy3/Cy5 ratios is an important factor to consider in choosing a normalization strategy. When normalization is carried out with selected HKGs, it is essential to use as many control genes as possible, particularly in a comparison between dissimilar samples. In the present invention, transcript levels of HKGs are divergent from sample to sample. For example, FIG. 6, shows scatter plots resulting from comparative hybridizations with the microarrays constructed with 96 HKGs in cells derived from the same origin (FIG. 6A) and from different origins (FIG. 6B). In FIG. 6A, comparisons of RNA sample derived from the same origin (Sample 1 and Sample 2) give similar expression levels of HKGs (Pearson's correlation coefficient, r=0.94). In contrast, in FIG. 6B, a comparison of RNA samples from dissimilar tissues (Sample 3 and Sample 2) give divergent expression levels of HKGs (r=0.70). Thus, inappropriate normalization may affect the results of selection of differentially expressed genes.

After the normalization process, the Cy3/Cy5 ratio for each individual spot is calculated against the normalized signal intensities. Then two separately scanned images of Cy3 and Cy5 are combined and pseudocolored to visualize the differentially expressed genes (refer to FIG. 2). Yellow spots indicate evenly expressed genes in both test and reference samples. Red and green spots denote up-regulated and down-regulated genes in the test compared to the reference sample, respectively.

Data Management Strategies.

Selection of differentially expressed genes. To date, various approaches have been attempted for the analysis and exploration of microarray data. However, investigators are confronted with the problem of deciding which expression ratios to regard as significant because there is no standard criteria for selection of differentially expressed genes. The most widely used method is the application of the cut-off value. Most studies have defined a 2- to 3-fold change in gene expression in the test sample compared with the reference as significant induction or repression (Fambrough et al., 1999, Cell, 97:727-741; Feng et al., 1999, Mol Endocrinol, 14:947-955; Zhao et al., 2000, Genes Dev, 14:981-993).

Differentially expressed genes can also be selected by calculating the confidence intervals (CIs). In this method, 99% CIs are typically used. A recent study showed that two strategies of fold changes and CIs were consistent. For example, 95% CIs corresponded to 1.5 fold change and 99% CIs corresponded to 2-fold changes, (Geiss et al., 2000, Virology, 266:8-16). However, these consistencies are not necessarily observed in biology, particularly when expression ratios show a deviated distribution such as that shown in FIG. 5B. Thus, similar to the normalization process for signal intensities, distribution of the expression ratios is an essential factor for choosing the appropriate gene selection strategy. In addition, ±2 or ±3 standard deviations of expression ratios are also used for selecting the differentially expressed genes (Karpf et al., 1999, Proc Natl Acad Sci, 96:14007-14012).

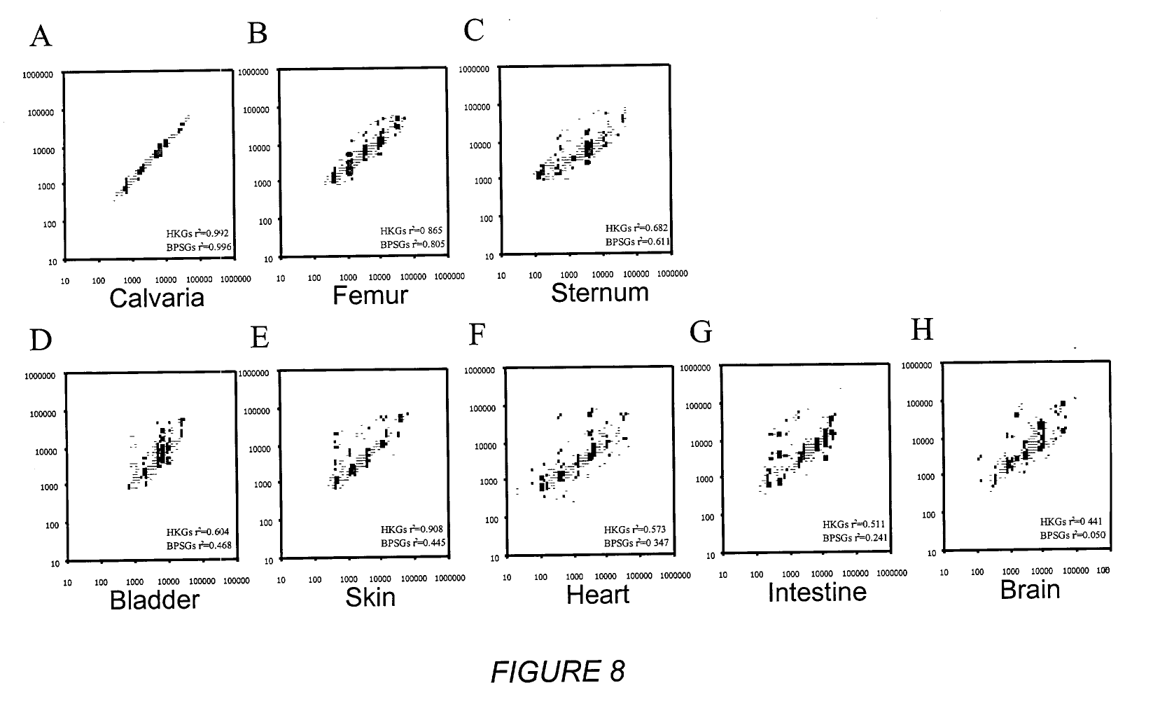

Data visualization and exploration. In order to visualize and explore microarray expression data, several methods are applied. For example, scatter plot analysis, similar to FIGS. 6-8, can identify outlying genes whose expression levels are different between the test and reference samples (Coller et al., 2000, Proc Natl Acad Sci, 97:3260-3265; Sudarsanam et al., 2000, Proc Natl Acad Sci, 97:3364-3369). By using one reference (e.g., one time-point) as a base line, the scatter plot comparisons of one reference with several test samples generate a Pearson correlation coefficient for each comparison (Khan et al., 1998, Cancer Res, 58:5009-5013; Voehringer et al., 2000, Proc Natl Acad Sci, 97: 2680-2685).

Several clustering methods have been applied for microarray data to identifying the sets of regulated genes. For example, K-means cluster, clustergram, and self-organizing maps with a software program make clustering of genes through several time points possible due to the similarity of their expression patterns (Eisen et al., 1998; Proc Natl Acad Sci, 95:14863-14868). Clustering analysis of sample-sample correlation can also be performed by the dendrogram method (Khan et al., 1998, Cancer Res, 58:5009-5013). In this technique, samples are clustered based on their gene expression profiles or their sensitivity to the stimuli, such as a drug, by measuring the distance metric of 1-Pearson correlation coefficient. Additionally, by adding a second dimension of clustering, such as gene clusters, to the dendrogram, a double dendrogram can be displayed (Perou et al., 1999; Proc Natl Acad Sci, 96:9212-9217). As another means of cluster analysis, genes can be classified into several categories based on their biological functions (Ferea et al., 1999, Proc Natl Acad Sci, 96:9721-9726; lyer et al., 1999, Science, 283:83-87; Ly et al., 2000, Science, 287:2486-2492). In addition, some investigators combine several clustering methods and/or other techniques to elucidate and explore the comprehensive and complex transcriptional regulation mechanisms and functional interactions of genes. As mentioned above, individual clustering techniques provide different information. Investigators, therefore, should choose or combine the appropriate methods for their purpose.

Other examples of data visualization include: Microarray data to visualize the chromosomal location of differentially expressed genes by histone H4 depletion (Wyrick et al., 1999, Nature, 402:418-421); ProbeBrowser software (http://molepi.stanford.edu/free_software.html), which integrates microarray data with the genomic positions of the hybridization targets and displays corresponding open reading frame annotations (Behr et al.,1999, Science, 284:1520-1523); and use of microarray has been used to determine the genetic network architecture by a combination of K-means clustering and sequence motif searching at the several time points throughout the yeast cell cycle (Tavazoie et al., 1999, Nat Genet, 22:281-285). These methods can visualize the relationship between differentially expressed gene and genomic region.

Biological validity of microarray data. To further ensure usefulness of microarray analysis in biology, comparisons with Northern blotting and RT-PCR have been performed. Results from microarray analysis is reliably consistent in comparison to Northern blotting and RT-PCR (Coller et al., 2000, Proc. Natl. Aca. Sci., 97:3260-3265). In another study, laser microdissection and the corresponding gene expression capture the in situ hybridization of positive cells by microarray tested. Two independent experiments validated the microarray data (Luo et al., 1999, Nat Med, 5:117-122). Thus, the high reliability of the microarray data has been documented.

Modifications of the Microarray.

Custom microarrays can be fabricated with any design depending on their purpose and question. Microarray design can be classified into two major categories: 1) Large-scale or versatile-type microarray; and 2) focused microarray.

Large-scale microarrays include thousands of target genes and are utilizable for any type of gene expression analysis because they contain different kinds of genes. In the present invention, the large-scale microarray is also referred to as the versatile-type microarray. This type of microarray is most common and has been used in genomic-wide research, mutational analyses, pharmacology, toxicology, aging research, molecular analyses of malignant tumors and other diseases. The results of these studies demonstrate the value of the versatile-type microarray for analysis of development, disease, and drug discovery at the transcriptional level.

A focused microarray, as its name implies, is designed for a specific purpose. A microarray in this category is constructed with selected genes of interest, or genes that are significant to a certain disease. For example, a focused microarray fabricated with approximately 96 inflammatory-related genes is used to evaluate the mRNA expression levels in samples from rheumatoid arthritis patients (Hellar et al., 1997, Proc Natl Acad Sci, 94:2150-2155). Another focused microarray with 148 target genes, including metabolic enzymes, DNA repair enzymes, stress proteins and cytokines is generated in order to analyze genetic response to toxicants (Bartosewicz et al., 2000, Arch Biochem Biophys, 376:66-73). Several other studies have reported combination analyses using the microarray and other differential display techniques. For instance, 26 differential immuno-absorption products of human glioblastoma (GBM) and normal brain tissues are used to construct a focused microarray for monitoring transcript levels in tumorous and non-tumorous brain specimens (Liau et al., 2000, Cancer Res, 60:1353-1360).

Furthermore, a focused microarray can be constructed on a smaller scale. An advantage of the smaller microarrays is that it is possible to reduce the time and cost for microarray fabrication, to minimize the RNA sample volume, and to maintain a high quality of target DNA. Although there is more limited data acquired with a focused microarray, it is a valuable tool for achieving specific objectives. Therefore, modifications of a standard microarray with due consideration given to their purpose are not outside the broad scope of the present invention.

Lastly, another type of microarray construction has been proposed. It is a cDNA library derived from a specific tissue, or “tissue-specific microarray”. For example, microarrays using rat heart cDNA libraries are fabricated to examine the gene expression profile in response to myocardial infarction (Sehl et al., 2000, Circulation, 101:1990-1999). In addition, others have fabricated microarrays with genomic DNA. Another microarray constructed with clones from chromosome 20 is used to analyze the DNA copy number variation in breast cancer (Pinkel et al., 1998, Nat Genet, 20:207-211). Using this approach, the result has demonstrated chromosome 20 aberrations in breast cancer.

Still in other experiments, microarray analysis has successfully identified gene amplifications and deletions throughout the genome (Pollack et al., 1999, Nat Genet, 23:41-46). These types of genomic analyses are useful for elucidating the pathological mechanisms of congenital and developmental abnormalities, including cleft lip and palate, and mandibular prognathism. Thus, various adaptations of microarray technology make it possible to analyze the genetic pathways and dynamic interactions of genes in various diseases including oral mucosal disease, premalignant and malignant tumors, periodontal disease, endodontic disease, temporomandibular joint disorders, cystic diseases and other normal and abnormal development of oral and craniofacial structures

Thus, the concept of functional genomics is a reality including global gene expression analysis by microarray technology, proteomics or a large-scale analyses of proteins, and computational biology (Dhand, 2000, Nature, 405). It is not outside the scope of this invention to use a double-stranded DNA microarray to analyze DNA-protein interactions (Bulyk et al., 1999, Nat Biotechnol, 17:573:577), or the differential-display proteomics assay using a protein chip (Pandey and Mann, 2000, Nature, 405:837-846). Thus, future post-genomic studies include functional genomics, global expression monitoring for genes and proteins and gene network analyses that combine several genetic analysis techniques.

In the present invention, 50 PSGs, for example, ECM genes, have been identified, cloned and sequenced. Extracellular matrix genes as discussed above are excellent molecular markers to assess, monitor and/or determine the phenotypes of cells.

More particularly, in the present invention, PSGs function as nucleotide targets on a microarray, and are utilized to assess, monitor and/or determine the phenotype of cells originally derived from stem cells of various tissues including adipose tissue, embryonic stem cells, embryonic germ cells, fetal stem cells and adult stem cells.

Specifically, at least 25 PSGs, for example, ECM genes, should be fabricated on any microarray to analyze gene expression profiles of cells. However, as many as possible that can be fabricated on any one microarray is preferred.

Also, in the present invention, HKGs are utilized as control nucleotide targets on similarly fabricated microarrays as the ECM targets. Many of the HKG nucleotide sequences are readily available through the National Institutes of Health GenBank database. House keeping genes as discussed above are constitutively expressed and show steady state levels of expression independent of the cell or tissue type.

Specifically, as many HKG nucleotide targets on a microarray as possible is preferred. However, in the present invention, a microarray consisting of 5,184 human genes, for example, human expressed sequence tags (ESTs), results in a higher background noise level, or a reduced signal to noise ratio. For example, 96 HKG nucleotide targets on a microarray results in good signal to noise ratio.

In general, the present invention, therefore, provides methods to diagnose cell phenotype by providing compositions (i.e. ECM gene and HKGs) that are fabricated on a microarray. These nucleotide targets are then hybridized to one or more fluorescently labeled cDNA probes made from RNA of tissues. Analysis of the fluorescence signals due to hybridization of probe and target provides a gene expression profile of those particular cells and/or tissues.

Furthermore, this method is used as a diagnostic tool to assess, monitor and/or determine cell phenotype. In the present invention, assessing, monitoring and/or determining cell phenotype of cells originally derived from stem cells is accomplished. However, the use of PSG and HKG nucleotide targets to diagnose cell and/or tissue phenotype of any origin is within the broad scope of this invention.

For example, the present invention also provides methods for diagnosis including assessing, monitoring and/or determining the phenotype of benign or malignant tumors, virus infected cells and/or tissues and tissue pathology.

The present invention by providing methods to assess, monitor and/or determine cell phenotype is much improved over current and existing methods of diagnosis including histological examination.

Accordingly, by way of example, but not a limitation, the following encompasses one or more embodiments of the invention. It is to be understood that the invention is not limited to these specific embodiments.

### EXAMPLE 1

Expression of genes associated with particular conditions. The gene expression of bone around titanium implants placed in the femurs of ovarectomized (OVX) and sham-operated rats is examined using a customized microarray with ECM-related genes. This microarray analysis demonstrated the differential expression of multiple ECM genes in the OVX rats. To date, expression of specific levels of ECM genes has not been shown.

### EXAMPLE 2

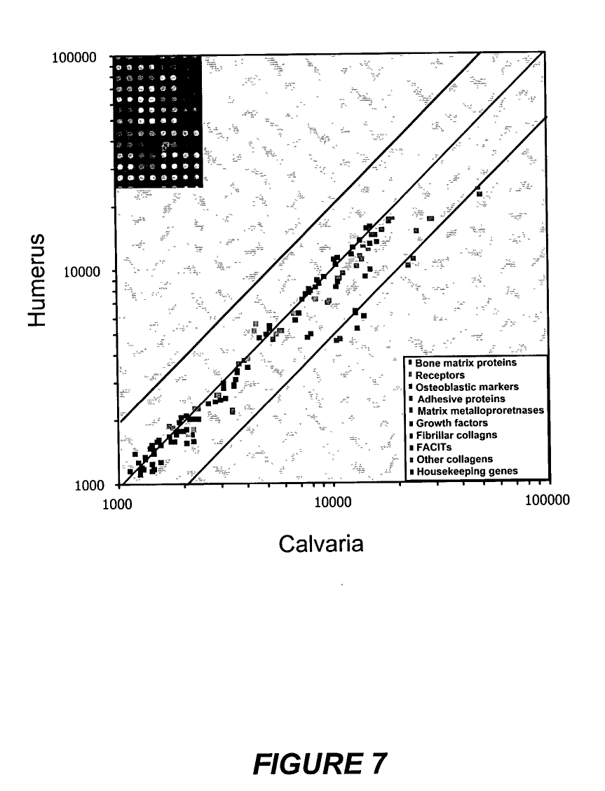

A customized microarray with 36 extracellular matrix(ECM)-related targets. FIG. 7 shows differential expression patterns of ECM genes between adult female mouse calvaria and that of humerus bones. In FIG. 7, ECM gene expression patterns are generally similar for both calvaria and humerus bone tissue. However, calvaria bone tissue has elevated expression (>2 fold) of collagen type1 alpha1; collagen type1 alpha2; collagen type9 alpha1; collagen type19 alpha1; and osteonectin.

### EXAMPLE 3

A focused microarray PSG cDNA microarray. A focused cDNA microarray containing approximately 50 PSGs, or in this example, approximately 50 ECM genes, and approximately 96 commonly expressed HKGs is fabricated using techniques described above. Table I and II list the full and abbreviated names of PSGs, or ECM genes, and HKGs, respectively.

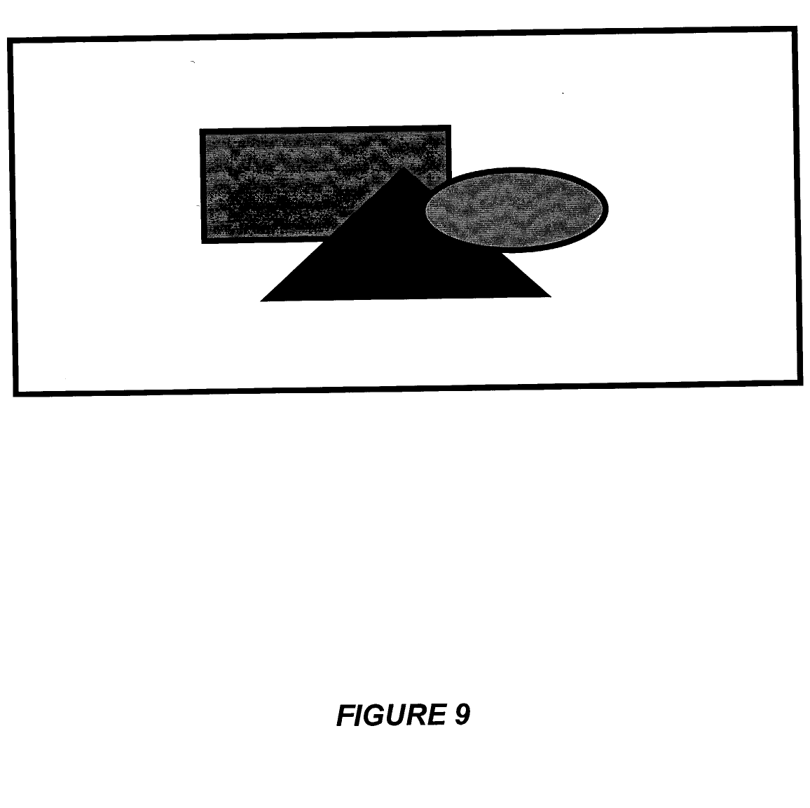

The focused cDNA microarray is fabricated to test the steady state expression of PSGs, and HKGs in mouse calvarial tissue as compared with that in mouse femur, sternum, bladder, skin, heart, intestine and brain tissue. As shown in FIG. 9, the results suggest that whereas HKGs maintain a strong correlation between similar and different tissues (Pearson's coefficient of correlation: r=0.72 to 0.93), PSGs exhibit sensitive differentiation correlation (r=0.49 to 0.90). The results show excellent Pearson's coefficient of correlation ranging from 0.72 to 0.87.

### EXAMPLE 4

A conventional microarray containing 5,184 human EST targets. To test upper limits of control genes or HKGs, a conventional cDNA microarray is fabricated using 5,184 human genes, in adipose-derived stem cells (ADSC) in control media (CM D12) or osteoblast differentiation medium (OS D12) from two different stromal vascular fractions (SVF). In FIG. 9, the results suggest that 5,184 human gene targets resulted in poor signal to noise ratio, or high background.

In summary, the above examples describe the usefulness of comprehensive gene expression profiling using microarray technologies, as well as the importance of using PSGs, for example, ECM-related genes, to assess, monitor and/or determine cell and tissue phenotype analysis.

Although the present invention has been described in terms of the preferred embodiment above, numerous modifications and/or additions to the above-described preferred embodiments would be readily apparent to one skilled in the art. Accordingly, the invention is not limited to the precise embodiments described in detail hereinabove.

We claim:

1. A composition for assessing, monitoring and/or determining a phenotype of cells or tissues comprising a plurality of nucleotide fragments, each of the nucleotide fragments encoding at least a portion of a phenotype specific gene.

2. A composition of claim 1 wherein the cells are derived from tissue-engineered cells.

3. A composition of claim 1 wherein the cells are derived from tissue containing stem cells including at least one cell type selected from a group consisting of embryonic stem cells, embryonic germ cells, fetal stem cells and adult stem cells.

4. A composition of claim 1 wherein the phenotype specific genes comprise at least 25 extracellular matrix genes selected from a group consisting of Osteocalcin, Osteopontin, Osteonectin, Alkaline phosphatase, Bone morphogenetic protein 7, Estrogen receptor, Vitamin D receptor, Bone morphogenetic protein 2, Core binding factor A1, Integrin alpha2, Integrin beta1, Integrin beta3, Parathyroid hormone receptor, Bone sialoprotein II, Matrix metalloproteinase 1, Matrix metalloproteinase 2, Laminin B1, Syndecan2, Chondroitin sulfate proteoglycan 1, Decorin, Fibronectin, Tenascin X, Collagen type1 alpha1, Collagen type1 alpha2, Collagen type2 alpha1, Collagen type3 alpha1, Collagen type4 alpha1, Collagen type4 alpha2, Collagen type5 alpha1, Collagen type5 alpha2, Collagen type6 alpha1, Collagen type6 alpha3, Collagen type7 alpha1, Collagen type9 alpha1, Collagen type9 alpha2, Collagen type9 alpha3, Collagen type10 alpha1, Collagen type11 alpha, Collagen type11 alpha1, Collagen type11 alpha2, Collagen type12 alpha1, Collagen type14 alpha1, Collagen type15 alpha1, Collagen type16 alpha1, Collagen type19 alpha1and Apolipoprotein E2.

5. A composition of claim 4 wherein the nucleotide fragments are affixed on an array.

6. A composition of claim 5 wherein the nucleotide fragments are no less than 20% of the total complete coding sequence of each of the extracellular matrix genes.

7. A composition of claim 5 wherein said nucleotide fragments are each about 100 to about 2000 nucleotides in length.

8. A composition of claim 5 wherein said nucleotide fragments are about 700 to about 1200 nucleotides in length.

9. A composition for assessing, monitoring and/or determining a phenotype of cells and tissues comprising a plurality of nucleotide fragments, each of the nucleotide fragments encoding at least a portion of a house keeping gene.

10. A composition of claim 9 wherein the cells are derived from tissue-engineered cells.

11. A composition of claim 9 wherein said house keeping genes comprise at least 50 genes selected from the group consisting of Human alpha-catenin, Human EST2, Human cytochrome c-1, Human uroporphyrinogen III synthase, HPV16 E1 binding protein, Human guanine nucleotide-binding (alpha subunit mRNA), Homo sapiens splicing factor SF3a120, Homo sapiens adenylyl cyclase-associated protein (CAP), Human cytochrome bc-1 complex core protein II, Human platelet-type phosphofructokinase, Homo sapiens deoxyhypusine synthase, Human hnRNP core protein A1, Human coatomer protein (HEPCOP), Homo sapiens phosphatidylinositol 4-kinase mRNA, Human AMP deaminase (AMPD2), Protein Translation Factor SUI1, Homo sapiens alpha centractin, Human Na/H antiporter (APNH1), Human lysosomal glycosylasparaginase (AGA), Human acidic ribosomal glycosylaparaginase (AGA), Human acidic ribosomal phosphoprotein P0, Human capping protein alpha, Human mercurial-insensitive water channel, Human ionizing radiation resistance conferring protein A, Human mitochondrial short-chain enoyl-CoA hydrase, Homo sapiens elongation factor-1-gamma, Homo sapiens catechol-O-methyltransferase (COMT), Human cytoplasmic beta-actin, Human calmodulin, Human 90 kDa heat shock protein, Human elongation factor Tu-mitochondrial, Human U1 snRNP-specific protein A, glycogenin-2 delta, Human Xq28, creatine transporter (SLC6A8), Human beta-tubulin, pulmonary surfactant protein (SP5), Human protein kinase, Homo sapiens cyclin H assembly factor, Human eIF-2-associated p67, Human liver glutamate dehydrogenase, Human superoxide dismutase (SOD-1), Human mitochondrial ADP/ADT translocator, Human eukaryotic initiation factor 2B-epsilon, Human chromatin assembly factor-I p60, Spermidine/spermine N1-acetyltransferase mRNA, Human translational initiation factor 2 beta subunit (eIF-2-beta), Human dihydrolipoamide dehydrogenase, Human cytoplasmic chaperonin hTRiC5, Homo sapiens protein tyrosine kinase (Syk), Human ADP/ATP translocase, T, Human ubiquitin-activating enzyme E1 (UBE1), Human cytochrome c oxidase subunit, Human histidyl-tRNA synthetase (HRS), Human topoisomerase I, Human 26S proteasome subunit p97, Human nuclear ribonucleoprotein particle (hnRNP) C protein, Human eukaryotic initiation factor 4Aii, Human lactate dehydrogenase-A (LDH-A, EC 1.1.1.27), Human sterol 27-hydroxylase (CYP27) mRNA, Human glutamate receptor 2 (HBGR2), Human alpha-2-macroglobulin mRNA, Human MRL3 ribosomal protein L3, Human histone H2B.1, Human chaperonin protein (Tcp20), Homo sapiens DNA (cytosin-5)-methyltransferase, Human eukaryotic initiation factor 4A1, Human heterogenous nuclear ribonucleoprotein D (hnRNP D), Human ADP-ribosylation factor 1 (ARF1), Homo sapiens endothelin-1 (EDN1), Human ADP-ribosylation factor, Human glyceraldehyde 3-phosphate dehydrogenase, Human mRNA (HA0643) for ORF, Homo sapiens cadherin-13, Human aminoacylase-1 (ACY1), Human DNA repair helicase (ERCC3), Human mitochondrial 3-ketoacyl-CoA thiolase beta-subunit of trifunctional and Human malate dehydrogenase (MDHA).

12. A composition of claim 11 wherein the nucleotide fragments are affixed on an array.

13. A composition of claim 12 wherein the nucleotide fragments are no less than 20% of the total complete coding sequence of each of the house keeping genes.

14. A composition of claim 12 wherein said nucleotide fragments are about 200 to about 2000 nucleotides in length.

15. A composition of claim 12 wherein said nucleotide fragment is about 700 to about 1200 nucleotides in length.

16. A method for assessing, monitoring and/or determining a phenotype of cells, the method comprising hybridizing cDNA probes made from the RNA of the cells to a plurality of DNA target genes on an array; and detecting hybridized cDNA probes on the array.

17. The method of claim 16 wherein the cells are derived from tissue-engineered cells.

18. The method of claim 16 wherein the RNA of the tissue is from dental, oral or maxillofacial tissue, and is originally derived from cell type selected from a group consisting of embryonic stem cells, embryonic germ cells, fetal stem cells and adult stem cells.

19. The method of claim 14 wherein the array is a cDNA microarray, an oligonucleotide microarray, a focused microarray, or a tissue-specific microarray.

20. The method of claim 16 wherein the DNA targets on an array comprise a plurality of phenotype specific genes selected from a group consisting of at least 25 extracellular matrix genes including Osteocalcin, Osteopontin, Osteonectin, Alkaline phosphatase, Bone morphogenetic protein 7, Estrogen receptor, Vitamin D receptor, Bone morphogenetic protein 2, Core binding factor A1, Integrin alpha2, Integrin beta1, Integrin beta3, Parathyroid hormone receptor, Bone sialoprotein II, Matrix metalloproteinase 1, Matrix metalloproteinase 2, Laminin B1, Syndecan2, Chondroitin sulfate proteoglycan 1 , Decorin, Fibronectin, Tenascin X, Collagen type4 alpha1, Collagen type4 alpha2, Collagen type2 alpha1, Collagen type3 alpha1, Collagen type4 alpha1, Collagen type4 alpha2, Collagen type5 alpha1, Collagen type5 alpha2, Collagen type6 alpha1, Collagen type6 alpha3, Collagen type7 alpha1, Collagen type9 alpha1, Collagen type9 alpha2, Collagen type9 alpha3, Collagen type10 alpha1, Collagen type1 alpha, Collagen type1alpha1, Collagen type11 alpha2, Collagen type12 alpha1, Collagen type14 alpha1, Collagen type15 alpha1, Collagen type16 alpha1, Collagen type19 alpha1and Apolipoprotein E2.

21. The method of claim 16 wherein said DNA targets on an array comprise at least 50 house keeping genes selected from a group consisting of Human alpha-catenin, Human EST2, Human cytochrome c-1, Human uroporphyrinogen III synthase, HPV16 E1 binding protein, Human guanine nucleotide-binding (alpha subunit mRNA), Homo sapiens splicing factor SF3a120, Homo sapiens adenylyl cyclase-associated protein (CAP), Human cytochrome bc-1 complex core protein 11, Human platelet-type phosphofructokinase, Homo sapiens deoxyhypusine synthase, Human hnRNP core protein A1, Human coatomer protein (HEPCOP), Homo sapiens phosphatidylinositol 4-kinase mRNA, Human AMP deaminase (AMPD2), Protein Translation Factor SUI1, Homo sapiens alpha centractin, Human Na/H antiporter (APNH1), Human lysosomal glycosylasparaginase (AGA), Human acidic ribosomal glycosylaparaginase (AGA), Human acidic ribosomal phosphoprotein P0, Human capping protein alpha, Human mercurial-insensitive water channel, Human ionizing radiation resistance conferring protein A, Human mitochondrial short-chain enoyl-CoA hydrase, Homo sapiens elongation factor-1-gamma, Homo sapiens catechol-O-methyltransferase (COMT), Human cytoplasmic beta-actin, Human calmodulin, Human 90 kDa heat shock protein, Human elongation factor Tu-mitochondrial, Human U1 snRNP-specific protein A, glycogenin-2 delta, Human Xq28, creatine transporter (SLC6A8), Human beta-tubulin, pulmonary surfactant protein (SP5), Human protein kinase, Homo sapiens cyclin H assembly factor, Human eIF-2-associated p67, Human liver glutamate dehydrogenase, Human superoxide dismutase (SOD-1), Human mitochondrial ADP/ADT translocator; Human eukaryotic initiation factor 2B-epsilon, Human chromatin assembly factor-I p60, Spermidine/spermine N1-acetyltransferase mRNA, Human translational initiation factor 2 beta subunit (eIF-2-beta), Human dihydrolipoamide dehydrogenase, Human cytoplasmic chaperonin hTRiC5, Homo sapiens protein tyrosine kinase (Syk), Human ADP/ATP translocase, T, Human ubiquitin-activating enzyme E1 (UBE1), Human cytochrome c oxidase subunit, Human histidyl-tRNA synthetase (HRS), Human topoisomerase I, Human 26S proteasome subunit p97, Human nuclear ribonucleoprotein particle (hnRNP) C protein, Human eukaryotic initiation factor 4Aii, Human lactate dehydrogenase-A (LDH-A, EC 1.1.1.27), Human sterol 27-hydroxylase (CYP27) mRNA, Human glutamate receptor 2 (HBGR2), Human alpha-2-macroglobulin mRNA, Human MRL3 ribosomal protein L3, Human histone H2B.1, Human chaperonin protein (Tcp20), Homo sapiens DNA (cytosin-5)-methyltransferase, Human eukaryotic initiation factor 4A1, Human heterogenous nuclear ribonucleoprotein D (hnRNP D), Human ADP-ribosylation factor 1 (ARF1), Homo sapiens endothelin-1 (EDN1), Human ADP-ribosylation factor, Human glyceraldehyde 3-phosphate dehydrogenase, Human mRNA (HA0643) for ORF, Homo sapiens cadherin-13, Human aminoacylase-1 (ACY1), Human DNA repair helicase (ERCC3), Human mitochondrial 3-ketoacyl-CoA thiolase beta-subunit of trifunctional and Human malate dehydrogenase (MDHA).

22. A method for assessing, monitoring and/or determining a cell phenotype in a patient comprising:
(a) isolating a plurality of cells from the patient;
(b) making one or more cDNA probes from RNA of said cells from the patient;
(c) labeling one or more of said cDNA probes with one or more fluorescent labels;
(d) hybridizing the fluorescently labeled cDNA probes to an array comprising of phenotype specific genes including extracellular matrix protein genes;
(e) also hybridizing fluorescently labeled cDNA probes to an array comprising of house keeping genes; and
(f) comparing hybridization profiles of the targets and the probes to first phenotype specific genes, and second to house keeping genes, to provide an expression profile of the cells in the patient to assess, monitor, and/or determine the cell phenotype.

23. The method of claim 22 whereby comparing hybridization profiles comprises calculating a Pearson's Coefficient of Correlation and Confidence Intervals.

24. The method of claim 22 whereby said measured plurality hybridization of targets to probes is the transcript levels of genes.

25. The method of claim 22 whereby the patient is a human.

26. The method of claim 22 whereby the patient is an animal.
